(19) United States
(12) Patent Application Publication
(10) Pub. No.: US 2023/0213467 A1
(43) Pub. Date: Jul. 6, 2023

(54) GAS SENSOR COMPRISING ONE OR MORE SENSING WIRES

(71) Applicant: Instrumems, Inc., Los Altos, CA (US)

(72) Inventors: Gilad ARWATZ, Los Altos, CA (US); Mehmet KOPRU, Mountain View, CA (US); Aditya CHAVAN, Los Altos, CA (US); Xingchen SHAO, Sunnyvale, CA (US); Yong Han Noel KIM, Mountain View, CA (US)

(21) Appl. No.: 18/092,841

(22) Filed: Jan. 3, 2023

Related U.S. Application Data
(60) Provisional application No. 63/296,415, filed on Jan. 4, 2022.

(51) Int. Cl. G01N 27/12; G01N 33/00

(52) U.S. Cl. CPC G01N 27/128; G01N 33/0063

(57) ABSTRACT

Numerous embodiments of a gas sensor and associated methods are described. In one embodiment, a gas sensor comprises a single wire. The resistance of the wire is measured for different temperatures, or the current through the wire or the voltage across the wire is measured for a constant temperature, and a profile for the surrounding gases is generated, enabling the surrounding gases to be identified. In another embodiment, a gas sensor comprises a first wire and a second wire in close proximity, where the first wire is used to generate temperature conditions, and the resistance of the second wire is measured for the different temperature conditions, or the current through the wire or the voltage across the wire is measured for a constant temperature. A profile for the surrounding gases is generated, enabling the surrounding gases to be identified.

### PRIORITY CLAIM

This application claims priority from U.S. Provisional Pat. Application No. 63/296,415, filed on Jan. 4, 2022, and titled “Gas Sensor Comprising One Or More Sensing Wires,” which is incorporated by reference as if set forth herein.

### FIELD OF THE INVENTION

Numerous embodiments of a gas sensor and associated methods are described.

### BACKGROUND OF THE INVENTION

The ability to quickly detect the presence of certain gases is of critical importance in many different contexts. For example, in healthcare applications, it is important to be able to detect gases such as oxygen, carbon dioxide, nitrogen, and other gases. In other applications, it is important to detect gases that are harmful to humans, such as carbon monoxide, or gases that are flammable, such as the refrigerant R32.

The prior art includes a variety of sensors for detecting certain gases. However, there is an ongoing need for sensors that are faster, smaller, easier to construct, and more power-efficient.

What is needed is a gas sensor that is an improvement over the prior art in these respects.

### SUMMARY OF THE INVENTION

Numerous embodiments of a gas sensor and associated methods are described. In one embodiment, a gas sensor comprises a single wire. The resistance of the wire is measured for different temperatures, or the current through the wire or the voltage across the wire is measured while the wire is held at a constant temperature. A profile for the surrounding gases is generated, enabling the surrounding gases to be identified. In another embodiment, a gas sensor comprises a first wire and a second wire in close proximity, where the first wire is used to generate temperature conditions, and the resistance of the second wire is measured for the different temperature conditions, or the current through the wire or the voltage across the wire is measured while the first wire and the second wire are held at a constant temperature. A profile for the surrounding gases is generated, enabling the surrounding gases to be identified.

### BRIEF DESCRIPTION OF THE DRAWINGS

FIG. 1 depicts a gas sensor comprising a single wire.

FIG. 2 depicts an electrical schematic of the sensor of FIG. 1.

FIG. 3 depicts a gas detection method using the sensor of FIG. 1.

FIG. 4 depicts another gas detection method using the sensor of FIG. 1

FIG. 5 depicts a gas sensor comprising two wires.

FIG. 6 depicts an enlargement of the two wires in the sensor of FIG. 5.

FIG. 7 depicts an electrical schematic of the sensor of FIG. 5.

FIG. 8 depicts a gas detection method using the sensor of FIG. 5.

FIG. 9 depicts another gas detection method using the sensor of FIG. 5.

FIGS. 10A and 10B depict a gas sensor mounted in a chassis.

FIG. 11 depicts a testing system.

FIG. 12 depicts voltages measured across a cold wire with different surrounding gases.

FIG. 13 depicts voltages measured across a cold wire with different surrounding gases when a pulse is received by a hot wire.

FIG. 14A depicts voltages measured across a hot wire and a cold wire when the surrounding gases is air.

FIG. 14B depicts voltages measured across a hot wire and a cold wire when the surrounding gases have a composition of 50% R32.

### DETAILED DESCRIPTION OF THE INVENTION

### Single Sensor Embodiment

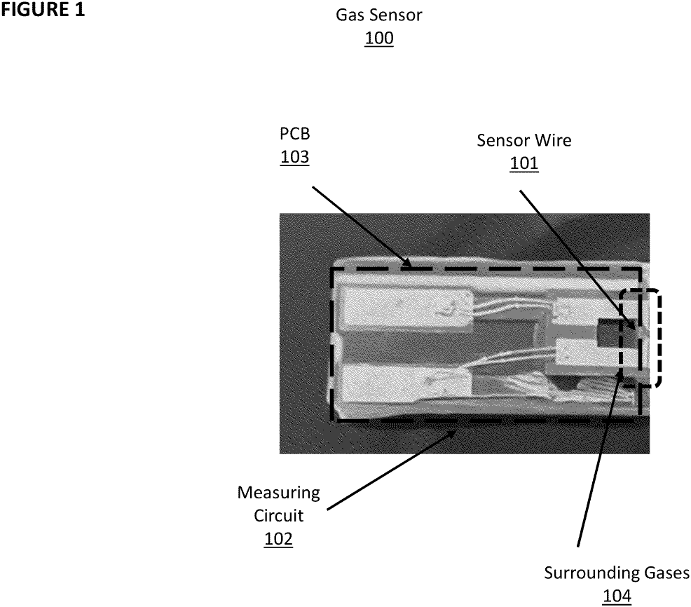

FIG. 1 depicts gas sensor 100. Gas sensor 100 comprises measuring circuit 102 mounted on PCB (printed circuit board) 103. Measuring circuit 102 comprises sensor wire 101. Sensor wire 101 is surrounded by surrounding gases 104. Surrounding gases 104 can comprise one or more pure gases of individual atoms (e.g., a noble gas such as neon), gases of elemental molecules of one type of atom (e.g., oxygen), gases of compound molecules made of a variety of atoms (e.g., carbon dioxide), or mixtures of any of these types of gases. For example, air contains nitrogen, oxygen, carbon dioxide, neon, and hydrogen.

In this embodiment, sensor wire 101 is a nanowire, which is a wire with a rectangular cross-section having a width of less than 5 microns and/or a height of less than 0.5 microns, the cross-section being taken perpendicular to a direction of current flow in measuring circuit 102 Sensor wire 101 may be of any suitable material with a non-zero Temperature Coefficient of Resistance (TCR), and a material commonly chosen is platinum with a positive TCR of 2000 -3920 ppm/°C, depending on purity, annealing, and other manufacturing steps. Other materials such as polysilicon may be used for sensor wire 101 where accuracy is not as much of a priority as cost.

The core principles of operation for gas sensor 100 are that:
(1) The resistance of sensor wire 101 changes in response to changes in its temperature, which mirror changes in the temperature of surrounding gases 104. The thermal conductivity of surrounding gases 104 depends upon the gas composition, which in turns affects the transfer of heat between sensor wire 101 and surrounding gases 1045. Therefore, by applying a well-controlled heat transfer between sensor wire 101 and surrounding gases 104, the measured Joule heating energy on the wire 101 will reflect the content of surrounding gases 104.
(2) If the temperature of surrounding gases 104 is controlled to remain constant, then the current through sensor wire 101 will be different depending on the heat transfer properties of surrounding gases 104. Different gases have different heat transfer properties due to differences in thermal conductivity. Therefore, by controlling the temperature of the system to remain constant, a change in current through sensor wire 101, or a change in voltage across sensor wire 101, will indicate a change in the content of surrounding gases.

Multiple embodiments of gas sensor 100 can be created that utilize these core principles.

Constant Voltage Embodiment: In one embodiment, measuring circuit 102 provides a Constant Voltage. A Constant Voltage circuit operates by providing a near constant voltage across sensor wire 101 and monitoring the change in resistance in sensor wire 101 to indicate and quantify the change in composition of surrounding gases 104. If a closed system containing surrounding gases 104 of Gas Composition X is heated with heat power P and a constant voltage V is applied to sensor wire 101, Gas Composition X will have a temperature gradient K1, and sensor wire 101 will have a first resistance gradientR1. If the surrounding gases 104 in closed system instead is Gas Composition Y and is heated with heat power P and a voltage V is applied to sensor wire 101, Gas Composition Y will have a temperature gradient K2, and sensor wire 101 will have a second resistance gradient R2 due to the difference in the heat transfer properties of Gas Compositions X and Y. The measured temperature gradients can be compared to a lookup table of known values for known gases to determine the content of surrounding gases 104. Here, a temperature gradient is the slope of temperature-time data points, and a resistance gradient is the slope of resistance-time data points. Alternatively, instead of determining a temperature gradient, a single temperature measurement can be taken by heating surrounding gases 104 with heat power P and applying a constant voltage V to sensor wire 101, and that temperature measurement can be compared to a lookup table of known values for known gases to determine the content of surrounding gases 104.

Constant Current Embodiment: In another embodiment, measuring circuit 102 provides a Constant Current. A Constant Current circuit operates by providing a near constant current to sensor wire 101 and monitoring the change in resistance in sensor wire 101 to indicate and quantify the change in composition of surrounding gases 104. If a closed system containing surrounding gases 104 of Gas Composition X is heated with heat power P and a constant current I is applied to sensor wire 101, Gas Composition X will have a temperature gradient K3, and sensor wire 101 will have a resistance gradient R3. If the surrounding gases 104 in closed system instead is Gas Composition Y and is heated with heat power P and a constant current I is applied to sensor wire 101, Gas Composition Y will have a temperature gradient K3, and sensor wire 101 will have a resistance gradient R4 due to the difference in the heat transfer properties of Gas Composition Y versus Gas Composition X. The measured temperature gradients can be compared to a lookup table of known values for known gases to determine the content of surrounding gases 104. Alternatively, instead of determining a temperature gradient, a single temperature measurement can be taken by heating surrounding gases 104 with heat power P and applying a constant current I to sensor wire 101, and that temperature measurement can be compared to a lookup table of known values for known gases to determine the content of surrounding gases 104.

Constant Temperature Embodiment: In another embodiment, gas sensor 100 provides a Constant Temperature. A Constant Temperature circuit operates by maintaining sensor wire 101 near a constant, elevated temperature and monitoring the change in power required to maintain constant temperature, where changes in power can be used to indicate and quantify the change in composition of surrounding gases 104. If a variable voltage is applied to sensor wire 101 in a closed system containing surrounding gases 104 of Gas Composition X to maintain a constant temperature K, sensor wire 101 will have a constant resistance R and will draw a varying current with gradient C1. If the surrounding gases 104 in closed system instead is Gas Composition Y and a variable voltage is applied to sensor wire 101 to maintain a constant temperature K, sensor wire 101 will have a constant resistance R and will draw a varying current with gradient C2. Here, a current gradient is the slope of current-temperature data points. The measured current gradients can be compared to a lookup table of known values for known gases under the same conditions to determine the content of surrounding gases 104. Alternatively, instead of determining a current gradient, a single current measurement can be taken while maintaining a constant temperature K in surrounding gases 104, and that current measurement can be compared to a lookup table of known values for known gases to determine the content of surrounding gases 104.

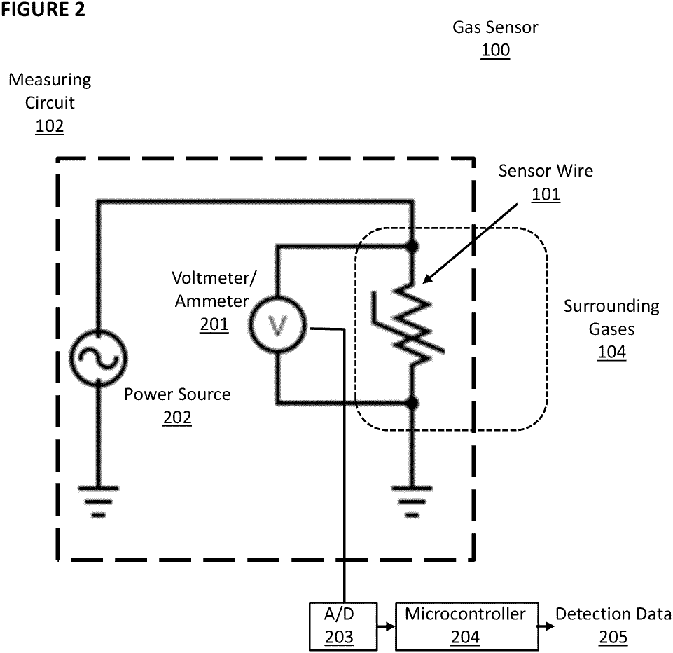

FIG. 2 depicts an electrical schematic of gas sensor 100. Gas sensor 100 implements the Constant Voltage or Constant Current scenarios described above. Gas sensor 100 as shown does not have a mechanism for keeping temperature constant and therefore does not implement the Constant Temperature scenario.

Measuring circuit 102 comprises voltmeter/ammeter 201, power source generator 202, and sensor wire 101. In one embodiment, power source 202 provides a constant voltage, and voltmeter/ammeter 201 measures current (which will change as the resistance of sensor wire 101 changes). In another embodiment, power source 202 provides a constant current, and voltmeter/ammeter measures voltage (which will change as the resistance of sensor wire 101 changes).

In the embodiment where power source 202 provides a constant voltage, power source 202 will apply a voltage, V1, to sensor wire 101. At time Ti, under Ohm’s law, the voltage across sensor wire 101 will be V_{Ti} = V1 = I_{Ti} * R_{Ti}, where R_{Ti} is the resistance of sensor wire 101 at time Ti and I_{Ti} is the current through sensor wire 101. The temperature, Kᵢ, of sensor wire 101 can be deduced since the materials composition of sensor wire 101 is known. A plurality of data points can be measured for voltage V1(for example, at times T1, T2, T3, etc.). Another plurality of data points can be measured for other constant voltages (V2, V3, etc.). As the voltage increases, the temperature of surrounding gases 104 will increase, and the resistance of sensor wire 101 will change. The thermal conductivity of a gas is a function of temperature as well as the content of the gas. The resulting sets of plurality of data points, which can be referred to as a profile, will be a unique signature for the exact content of surrounding gases 104. For example, the profiles of air and pure oxygen will be different.

Similarly, in a Constant Current embodiment, power source 202 will inject a current, I1, into sensor wire 101. At time Ti, under Ohm’s law, the voltage across sensor wire 101 will be V_{Ti} = I1 * R_{Ti}, where R_{Ti} is the resistance of sensor wire 101 at time Ti and V_{Ti} is the voltage across sensor wire 101. The temperature, Kᵢ, of sensor wire 101 can be deduced since the materials composition of sensor wire 101 is known. A plurality of data points can be measured for current I1 (for example, at times T1, T2, T3, etc.). Another plurality of data points can be measured for other constant currents (I2, I3, etc.). As the current increases, the temperature of surrounding gases 104 will increase, and the resistance of sensor wire 101 will change. The thermal conductivity of a gas is a function of temperature as well as the content of the gas. The resulting sets of plurality of data points, which can be referred to as a profile, will be a unique signature for the exact content of surrounding gases 104. For example, the profiles of air and pure oxygen will be different.

The thermal behavior of sensor wire 101 will now be described. More specifically, a nanowire with radius α with a large aspect ratio larger than 100 is simplified as an infinite line heat source. Through applying a constant power operation mode to sensor wire 101, a constant heat flux q is generated. The line source transfers heat radially into the surrounding gases 104 of an infinite incompressible fluid medium of constant density ρ, thermal conductivity k, and specific heat cₚ. The temperature history of sensor wire 101 is described by the equation:

$$Δ T \left(a , t\right) = \left(\frac{q}{4 π k}\right) I n \left(4 α t / {a}^{2} C\right)$$

where α is thermal diffusivity α = ^{k}/_{pCp}, and C is a mathematical constant. The system determines the thermal conductivity k by extracting the slope of ΔT versus In(t), since q is a known parameter in the measurement. The measured thermal conductivity can serve as a criterion for gas detection.

Optionally, the output of voltmeter/ammeter 201 is provided to analog-to-digital converter 203, which converts the measured voltage or current into digital form, which is then provided to microcontroller 204 for processing. Microcontroller 204 can generate detection data 205.

Detection data 205 can comprise an alert if microcontroller 204 detects a change in the composition of surrounding gases 104, which would be indicated by a change in the measured profiles of surrounding gases 104. An alert can comprise a visual alert (such as a light, a text message, an email, or other visual display) or an audible alert (such as a beep, siren, or synthesized or recorded speech).

Detection data 205 also can comprise an identification of the composition of surrounding gases 104 based on the data collected. For example, if a profile is obtained for surrounding gases 104 based on measurements obtained periodically while signal generator 202 generates a particular pattern, that profile can be compared to data stored in a lookup table and the composition of surrounding gases 104 can be determined based on the best fit in the lookup table.

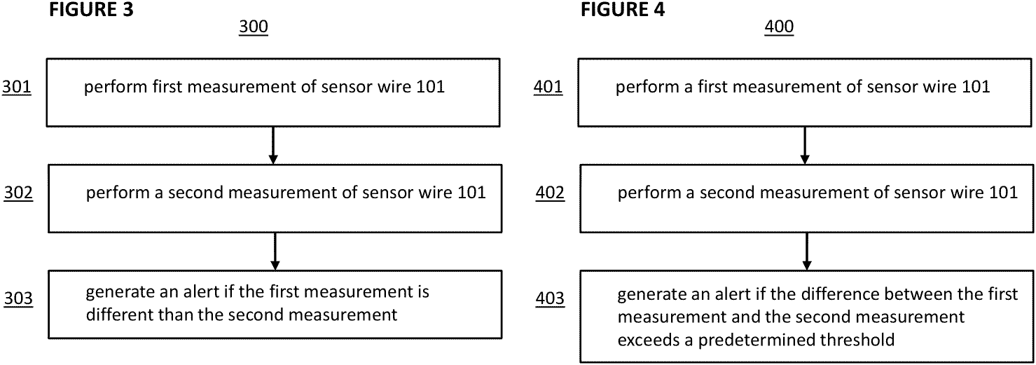

FIG. 3 depicts gas detection method 300 that can be performed by gas sensor 100. A first measurement is performed on sensor wire 101(step 301). The measurement can be of the voltage across sensor wire 101 when a constant current is injected into sensor wire 101 or it can be of the current through sensor wire 101 if a constant voltage is applied to sensor wire 101. A second measurement is performed on sensor wire 101 (step 302). An alert is generated if the first measurement is different than the second measurement (step 303). Here, each of the first measurement and the second measurement can be either a single measurement at a single point in time or a series of measurements at multiple points in time using a single constant voltage or constant current or multiple constant voltages or constant currents.

FIG. 4 depicts gas detection method 400 that can be performed by gas sensor 100. A first measurement is performed on sensor wire 101 (step 401). The measurement can be of the voltage across sensor wire 101 when a constant current is injected into sensor wire 101 or it can be of the current through sensor wire 101 if a constant voltage is applied to sensor wire 101 A second measurement is performed on sensor wire 101 (step 402). An alert is generated if the difference between the first measurement and the second measurement exceeds a predetermined threshold. (step 403). Here, each of the first measurement and the second measurement can be either a single measurement at a single point in time or a series of measurements at multiple points in time using a single constant voltage or constant current or multiple constant voltages or constant currents.

### Dual Sensor Embodiment

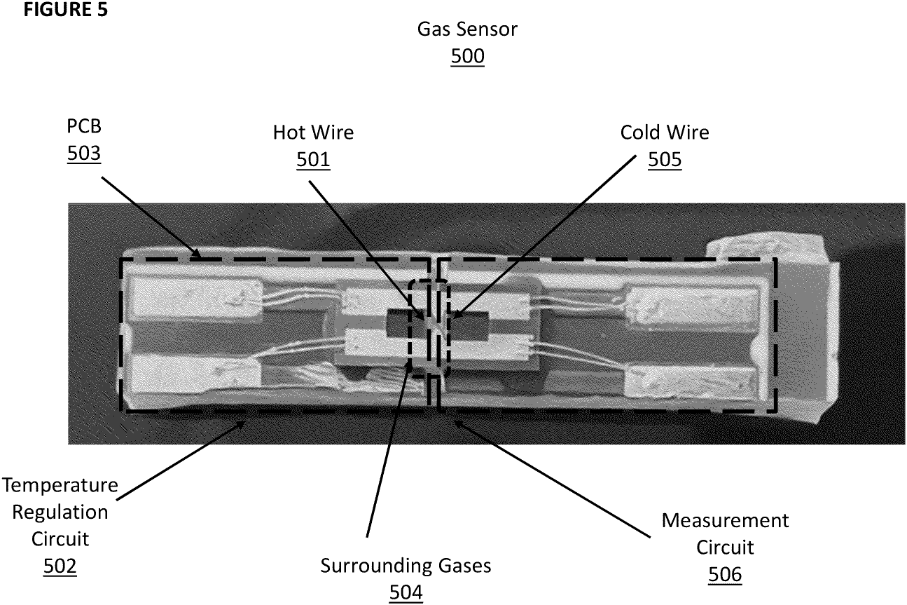

FIG. 5 depicts gas sensor 500. Gas sensor 500 comprises temperature regulation circuit 502 and measurement circuit 506 mounted on PCB 503. Temperature regulation circuit 502 comprises hot wire 501, and measurement circuit 506 comprises cold wire 505. Hot wire 501 and cold wire 505 are surrounded by surrounding gases 504. Here, cold wire 505 can be referred to as a sensor wire. Unlike the single sensor embodiment, the dual sensor embodiment is able to control the temperature of surrounding gases using temperature regulation circuit 502.

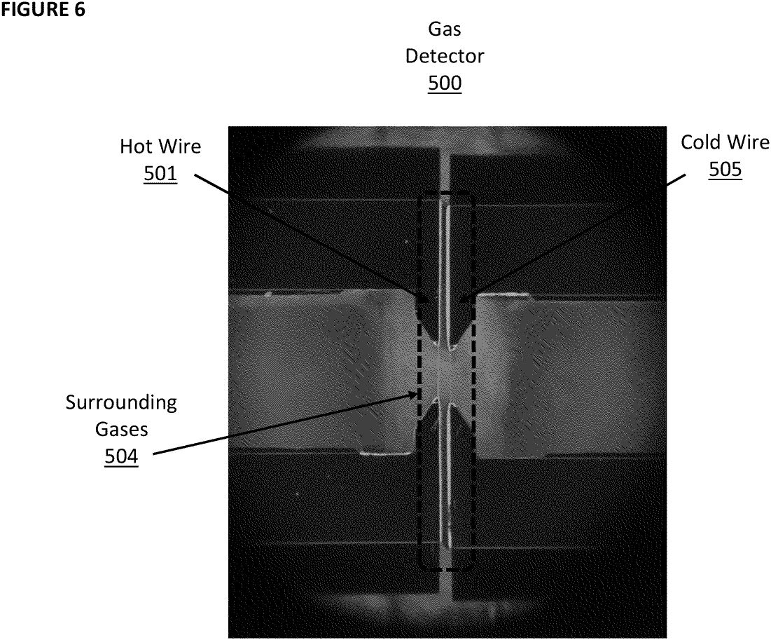

FIG. 6 depicts an enlarged view of hot wire 501 and cold wire 505 in gas sensor 500. In this embodiment, hot wire 501 and cold wire 505 are structurally identical wires in close physical proximity. However, it will be understood that hot wire 501 and cold wire 502 need not be identical. In this embodiment, hot wire 501 and cold wire 505 are nanowires, of the same type as sensor wire 101 in gas sensor 100 of FIGS. 1-4.

In this embodiment, hot wire 501 is used as a control mechanism to alter the temperature of surrounding gases 504. It can be appreciated that hot wire 501 could be replaced with another temperature control device.

The core principles of operation for gas sensor 500 is the same as those described above for gas sensor 100.

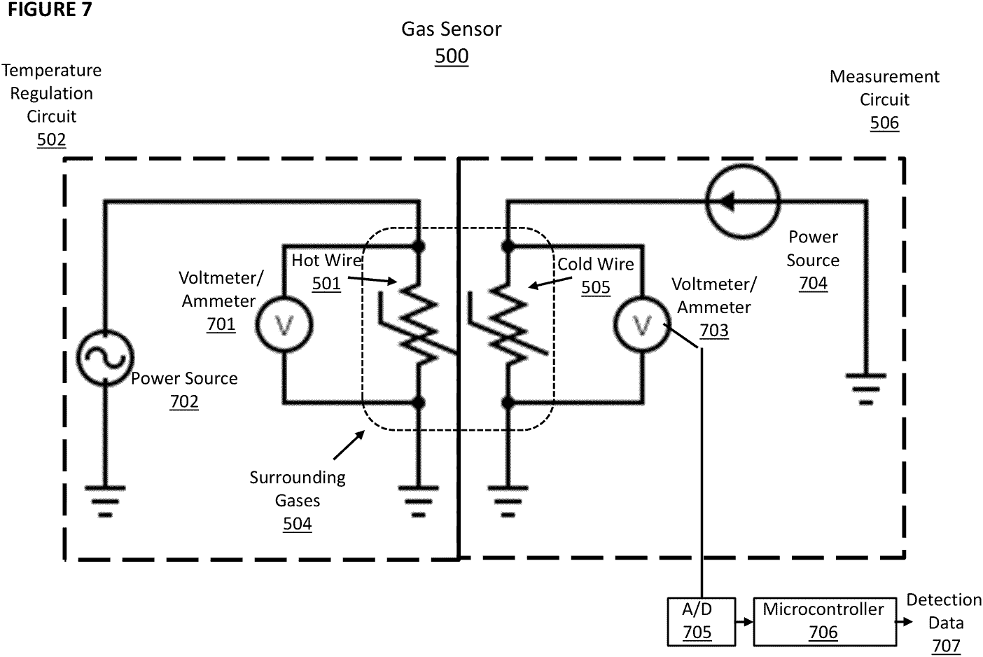

FIG. 7 depicts an electrical schematic of gas sensor 500. Temperature regulation circuit 502 comprises voltmeter/ammeter 701, power source 702, and hot wire 501. Measurement circuit 506 comprises voltmeter/ammeter 703, power source 704, and cold wire 505. Gas sensor 500 is able to operate in a Constant Voltage, Constant Current, or Constant Temperature mode.

In a Constant Voltage mode, power source 704 provides a constant voltage, V1, to cold wire 505. Voltmeter/ammeter 703 measures the current I_{Ti}, through cold wire 505 at time Ti, according to Ohm’s law, V1 = I_{Ti} * R_{Ti}. R_{Ti} is the resistance of cold wire 505 at time Ti, which will be affected by the temperature of surrounding gases 504 that in turn is affected by the heat generated by hot wire 501 in temperature regulation circuit 502. Data can be collected at one or more points of time (T1, T2, etc.). The constant voltage can be altered and data again collected as one or more points of time, thus creating a profile.

In a Constant Current mode, power source 704 provides a constant current, I1, to cold wire 505. Voltmeter/ammeter 703 measures the voltage, V_{Ti}, across cold wire 505 at time Ti, according to Ohm’s law, V_{Ti} = I1 * R_{Ti}. R_{Ti} is the resistance of cold wire 505 at time Ti, which will be affected by the temperature of surrounding gases 504 that in turn is affected by the heat generated by hot wire 501 in temperature regulation circuit 502. Data can be collected at one or more points of time (T1, T2, etc.). The constant current can be altered and data again collected as one or more points of time, thus creating a profile.

In a Constant Temperature mode, temperature regulation circuit 502 maintains surrounding gases 504 as a constant temperature, K1. Power source 704 provides a voltage, VTi, to cold wire 505, and voltmeter/ammeter 703 measures the current I_{Ti}, through cold wire 505 at time Ti, according to Ohm’s law, VTi = ITi * R1. R1 is the resistance of cold wire 505 at temperature K1. Data can be collected at one or more points of time (T1, T2, etc.). The constant temperature can be altered and data again collected at one or more points of time, thus creating a profile.

Optionally, the output of voltmeter 703 is provided to analog-to-digital converter 705, which converts the analog voltage or current into digital form, which is then provided to microcontroller 706 for processing. Microcontroller 706 generates detection data 707.

Detection data 707 can comprise an alert if microcontroller 706 detects a change in the composition of surrounding gases 504, which would be indicated by a change in the measured resistance of cold wire 505. An alert can comprise a visual alert (such as a light, a text message, an email, or other visual display) or an audible alert (such as a beep, siren, or synthesized or recorded speech).

Detection data 707 also can comprise an identification of the composition of surrounding gases 504 based on the data collected. For example, if a profile is obtained for surrounding gases 504 based on measurements obtained periodically while signal generator 702 generates a particular pattern, that profile can be compared to data stored in a lookup table and the composition of surrounding gases 504 can be determined based on the best fit in the lookup table for the measured profile.

In some embodiments, a more complex criteria can be used to indicate or quantify the composition of surrounding gases 505 and changes thereof. For instance, surrounding gases 504 can be characterized by the following conduction heat transfer and/or convective heat transfer equations:

Conductive Heat Transfer:

$$Q = - k A \frac{Δ T}{Δ {x}^{′}}$$

Where Q is the rate of heat transfer from cold wire 505 to surrounding gases 504, k is the conductive heat transfer coefficient, A is the surface area of cold wire 505, and

$$\frac{Δ T}{Δ x}$$

is the temperature gradient.

Convective Heat Transfer:

$$Q = h A \left({T}_{w} - {T}_{a}\right)$$

Where Q is the rate of heat transfer from cold wire 505 to surrounding gases 504, h is the convective heat transfer coefficient, A is the surface area of cold wire 505 where the heat transfer takes place, T_{w} is the temperature of cold wire 505, and T_{α} is the ambient temperature of surrounding gases 504. When operated in Constant Current, Constant Voltage, or Constant Temperature mode, the cold wire 505 signal will be directly related to power dissipation at the interface of cold wire 505 and surrounding gases 504, which is a function of Q. Thus, the change in signal when the composition of surrounding gases 504 changes can be correlated to a change in Q.

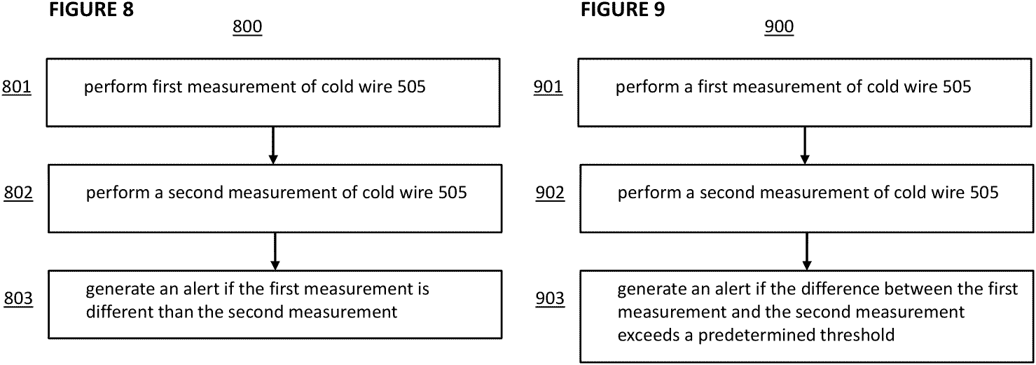

FIG. 8 depicts gas detection method 800 that can be performed by gas sensor 500. A first measurement is performed on cold wire 505 (step 801). The measurement can be of the current through cold wire 505 or the voltage across cold wire 505. A second measurement is performed on cold wire 505 (step 802). An alert is generated if the first measurement is different than the second measurement (step 803). Here, each of the first measurement and the second measurement can be either a single measurement at a single point in time or a series of measurements at multiple points in time using a single constant voltage, current, or temperature, or multiple different constant voltages, currents, or temperature.

FIG. 9 depicts gas detection method 900 that can be performed by gas sensor 500. A first measurement is performed on cold wire 505 (step 901). The measurement can be of the current through cold wire 505 or the voltage across cold wire 505. A second measurement is performed on cold wire 505 (step 902). An alert is generated if the difference between the first measurement and the second measurement exceeds a predetermined threshold. (step 903). Here, each of the first measurement and the second measurement can be either a single measurement at a single point in time or a series of measurements at multiple points in time using a single constant voltage, current, or temperature, or multiple different constant voltages, currents, or temperature.

### Chassis

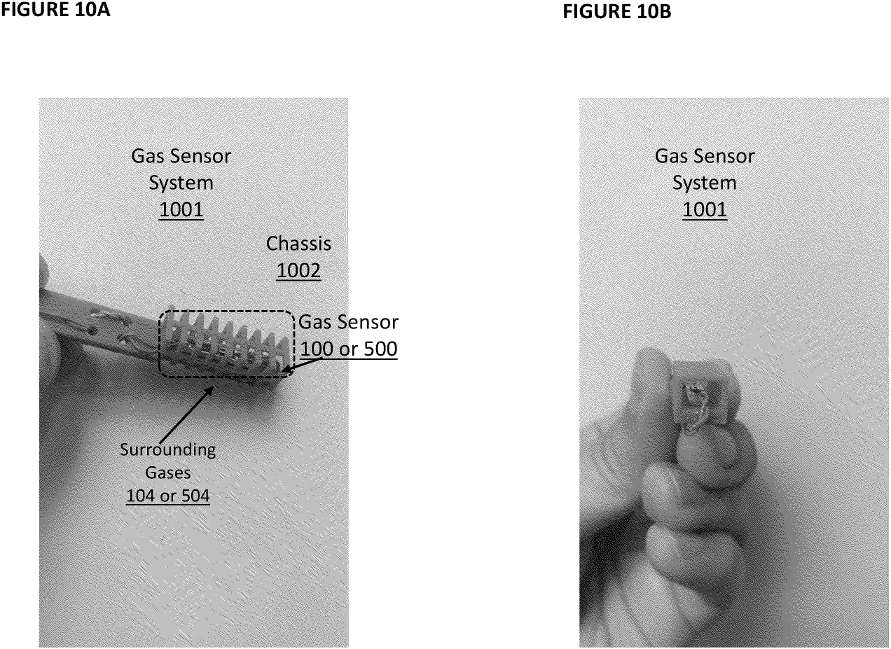

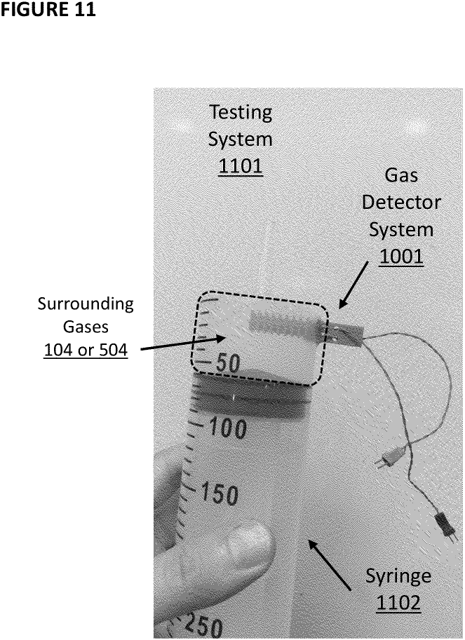

FIGS. 10A and 10B depict mechanical aspects used in conjunction with gas sensor 100 or 500. In FIG. 10A, gas sensor 100 or 500 is mounted in chassis 1002. Chassis 1002 provides physical protection to gas sensor 100 or 500 yet allows surrounding gases 104 or 504 to come into contact with gas sensor 100 or 500. The combination of gas sensor 100 or 500 mounted in chassis 1002 can be referred to as gas sensor system 1001. FIG. 10B depicts a side view is shown of gas sensor system 1001.

### Testing System

FIG. 11 depicts an embodiment of testing system 1101 that can be used with gas sensor 100, gas sensor 500, or gas sensor system 1001. In the example shown, gas sensor system 1001 is placed into syringe 1102 in an airtight manner.

### Exemplary Data

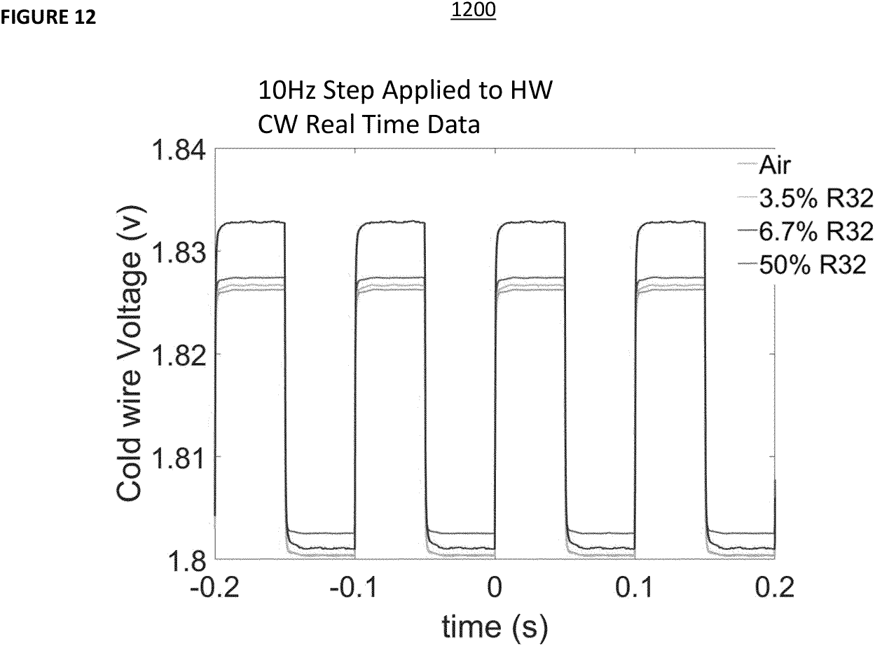

FIGS. 12-14 depict data collected by the applicant through testing of the disclosed embodiment of gas sensor 500.

FIG. 12 depicts graph 1200. A square pulse is provided to hot wire 501, which affects the temperature of surrounding gases 504 and consequently of cold wire 505. Four sets of data are depicted - one where surrounding gases 504 comprises air, one where surrounding gases 504 comprises a 3.5% concentration of R32, one where surrounding gases 504 comprises a 6.7% concentration of R32, and one where surrounding gases 504 comprises a 50% concentration of R32. As can be seen, the measured value (here, voltage) across cold wire 505 varies as the composition of surrounding gases 504 changes, which is a result of the resistance of cold wire 505 changing as its rate of heat dissipation changes as the composition of surrounding gases 504 changes.

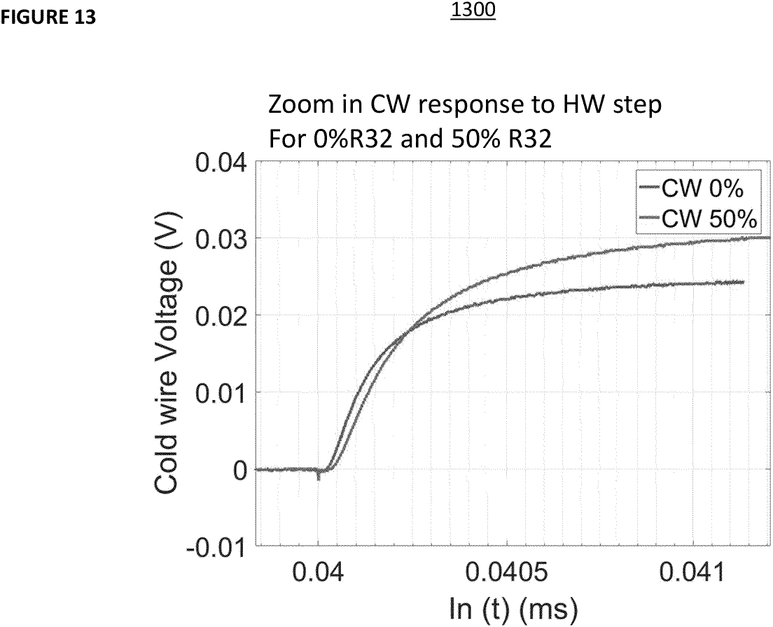

FIG. 13 depicts graph 1300. Graph 1300 shows the measured value (here, voltage) of cold wire 405 in response to a pulse in hot wire 401 in the situation where surrounding gases 404 is 0% R32 and in the situation where surrounding gases 504 is 50% R32. As can be seen, cold wire 505 displays a difference response in the two situations due to the difference in thermal conductivity of surrounding gases 504.

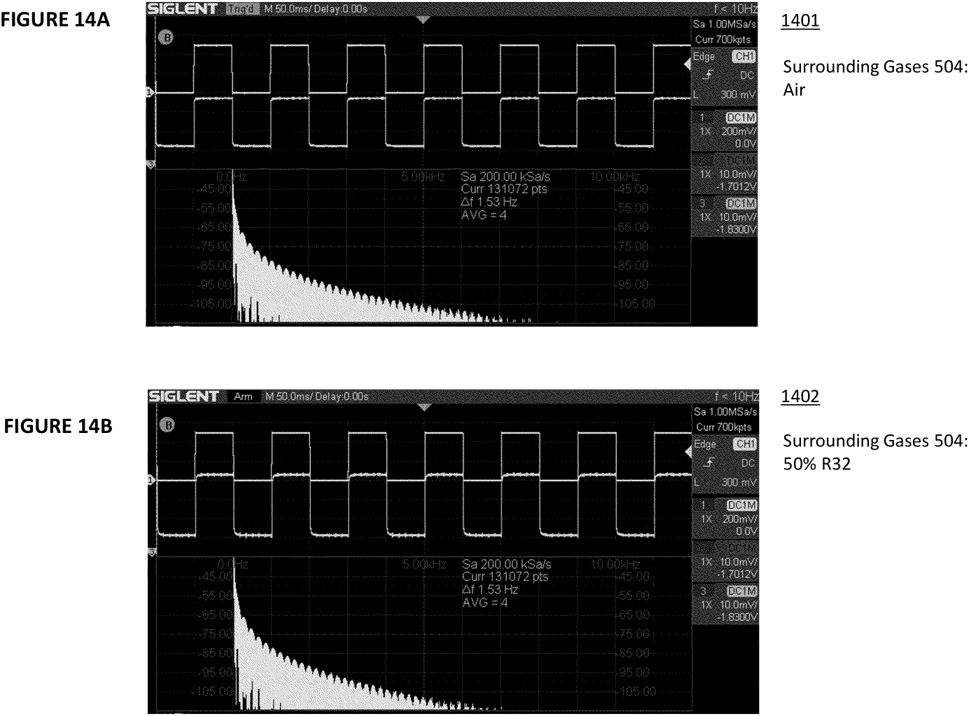

FIGS. 14A and 14B depict graphs 1401 and 1402, respectively. In FIG. 14A, surrounding gases 504 is air, and in FIG. 14B, surrounding gases 504 has a 50% concentration of R32.

Graphs 1401 and 1402, show the square-wave frequency response calculated by taking the Fourier Transform of the measured voltage across cold wire 505. As can be seen, the frequency response of the system differs based on the composition of surrounding gases 504. This unique pattern in the Fourier Transform can be measured by applying a voltage of one or more frequencies on hot wire 501 and measuring the response of cold wire 505. Any change in the response can be used to indicate that a change in gas composition of surrounding gases 504 has occurred, possibly signaling a hazardous condition.

It should be noted that, as used herein, the terms “over” and “on” both inclusively include “directly on” (no intermediate materials, elements or space disposed therebetween) and “indirectly on” (intermediate materials, elements or space disposed therebetween). Likewise, the term “adjacent” includes “directly adjacent” (no intermediate materials, elements or space disposed therebetween) and “indirectly adjacent” (intermediate materials, elements or space disposed there between), “mounted to” includes “directly mounted to” (no intermediate materials, elements or space disposed there between) and “indirectly mounted to” (intermediate materials, elements or spaced disposed there between), and “electrically coupled” includes “directly electrically coupled to” (no intermediate materials or elements there between that electrically connect the elements together) and “indirectly electrically coupled to” (intermediate materials or elements there between that electrically connect the elements together). For example, forming an element “over a substrate” can include forming the element directly on the substrate with no intermediate materials/elements therebetween, as well as forming the element indirectly on the substrate with one or more intermediate materials/elements there between.

What is claimed is:

1. A gas sensor, comprising:
a nanowire, wherein a measured value of the nanowire changes in response to a change in temperature of the nanowire; and
a measuring circuit coupled to the nanowire for identifying changes in the measured value.

2. The gas sensor of claim 1, wherein the measured value is resistance.

3. The gas sensor of claim 1, wherein the measured value is voltage.

4. The gas sensor of claim 1, wherein the measured value is current.

5. The gas sensor of claim 1, wherein the measured value is power consumption.

6. The gas sensor of claim 1, further comprising:
an analog-to-digital converter for converting an analog output from the measuring circuit into digital data.

7. The gas sensor of claim 6, further comprising:
a microcontroller for receiving the digital data.

8. The gas sensor of claim 7, wherein the microcontroller is configured to generate an alert when a change in resistance of the nanowire is detected.

9. The gas sensor of claim 8, wherein the microcontroller is configured to generate an alert when a plurality of measured values of the nanowire match a predetermined pattern.

10. The gas sensor of claim 1, further comprising:
a variable temperature source for changing the temperature of surrounding gases around the nanowire.

11. The gas sensor of claim 10, wherein the variable temperature source comprises a second nanowire.

12. A method of sensing a gas, comprising:
measuring a value of a nanowire, wherein the value changes in response to a change in temperature of the nanowire; and
identifying changes in the measured value.

13. The method of claim 12, wherein the value is resistance.

14. The method of claim 12, wherein the value is voltage.

15. The method of claim 12, wherein the value is current.

16. The method of claim 12, wherein the value is power consumption.

17. The method of claim 12, further comprising:
generating a digital output in response to the changes in the value.

18. The method of claim 17, further comprising:
generating an alert when a change in the value is detected.

19. The method of claim 18, further comprising:
generate an alert when a plurality of measured values of the nanowire match a predetermined pattern.

20. The method of claim 12, further comprising:
changing, by a variable temperature source, the temperature of surrounding gases around the nanowire.

21. The method of claim 20, wherein the variable temperature source comprises a second nanowire.
